SEQUENCE LISTING (5 sequences)

SEQ ID NO: 1 - protein, 12 aa, synthetic construct
GAHWQFNALTVR

SEQ ID NO: 2 - protein, 15 aa, synthetic construct
STMMSRSHKTRSHHV

SEQ ID NO: 3 - protein, 12 aa, synthetic construct
YKQKIKHVVKLK

SEQ ID NO: 4 - protein, 11 aa, synthetic construct
RYPISRPRKRC

SEQ ID NO: 5 - protein, 27 aa, synthetic construct
LKQKIKHVVKLKVVVKLRSQLVKRKQN

(19) United States
(12) Patent Application Publication
(10) Pub. No.: US 2025/0049885 A1
(43) Pub. Date: Feb. 13, 2025

(54) HYALURONIC ACID BINDING PEPTIDE AS A THERAPEUTIC TO CONTROL FIBROBLAST ACTIVATION AND FIBROTIC CAPSULE FORMATION

(71) Applicant: SOUTH DAKOTA BOARD OF REGENTS, Pierre, SD (US)

(72) Inventors: TUGBA OZDEMIR, Rapid City, SD (US); BETH BLAKE, Rapid City, SD (US)

(21) Appl. No.: 18/782,811

(22) Filed: Jul. 24, 2024

Related U.S. Application Data
(60) Provisional application No. 63/515,211, filed on Jul. 24, 2023.

(51) Int. Cl. A61K 38/17; A61K 47/54; A61L 26/00; A61L 27/10; A61L 27/34

(52) U.S. Cl. CPC A61K 38/1709; A61K 47/54; A61L 26/0004; A61L 26/0047; A61L 27/10; A61L 27/34; A61L 2300/252; A61L 2400/18

(57) ABSTRACT

Articles, such as implants and medical devices, treated with a hyaluronic acid binding peptide are provided. Articles are coated with a surface functionalizing agent which bind to and immobilize the hyaluronic acid binding peptide. Methods of improving and/or reducing foreign body reaction to an article are also provided, as are methods of decreasing inflammation, promoting wound healing, and treating dermal conditions.

### CROSS-REFERENCE TO RELATED APPLICATIONS

This application claims priority under 35 U.S.C. § 119 to provisional application Ser. No. 63/515,211, filed Jul. 24, 2023, herein incorporated by reference in its entirety.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on May 8, 2024, is named “P14438US01_SequenceListing.xml” and is 5.515 bytes in size.

### TECHNICAL FIELD

This disclosure relates to articles treated with hyaluronic acid and methods for reducing and/or preventing fibrotic capsule formation and foreign body reaction.

### BACKGROUND

Fibrosis is a phenomenon that occurs when there is excess extracellular matrix (ECM) deposition that leads to the formation of collagenous scar tissue in place of normal tissue and is largely associated with inflammation. Fibrosis has a role in several clinical problems such as hypertrophic scar formation, liver cirrhosis. Chron's disease, and issues with biomedical implants. In cases involving implants, this can lead to fibrotic capsule formation (FCF). The fibrotic capsules form under chronic inflammation conditions that result from the body treating the implant as a foreign substance. The macrophages present at the site attempt to break down the implant, and upon failure, foreign body giant cells (FBGCs) form. They also try to break down the implant but will ultimately form a fibrous capsule around the implant to isolate it from the body as a protective measure. With the exponential increase of implantable biomaterials in an aging population, it is expected that FCF will be a challenge to overcome.

The formation of a fibrotic capsule around an implant has several causes: among them, incompatibility with the body leading to prolonged inflammation. As described above. FBGCs form out of macrophage fusion during the body's initial attempt to break down the foreign material. This reaction can often be attributed to incompatibility between the body and the implant. Many implants are made of metal, or other materials not natural to the body, which can have varying degrees of biocompatibility. Several of these materials are bioinert, meaning that they neither interact with the body, nor encourage a negative reaction upon implantation. However, should the implant be incompatible, the foreign body reaction (FBR) will occur. During the FBR, the FBGCs will form and adhere to the material surface and undergo frustrated phagocytosis, where they will begin to release degradation mediators. Polymer materials are more likely to breakdown under this process, but other materials, like metals and silicone, will withstand and become encapsulated by fibrous tissue.

The impact of fibrosis can be far reaching as it can affect implant functionality and disease progression. In implants, the formation of a fibrotic capsule can lead to chronic inflammation, reduced functionality of the implant, or even complete implant failure. Thus, there exists a need in the art for improved articles, biomaterials, and methods which reduce and/or prevent foreign body reaction and fibrotic capsule formation.

### SUMMARY

Disclosed herein are articles treated with hyaluronic acid binding peptide (HABP). The disclosure also provides for methods of treating the articles with HABP. The treated articles provide various advantages over untreated articles. The advantages and benefits of the disclosed treated articles and related methods are described herein.

The present disclosure provides articles treated with hyaluronic acid binding peptide (HABP). In certain embodiments, the article comprises a coating of a surface functionalizing agent on a surface of the article, wherein a hyaluronic acid binding peptide (HABP) is bound to the surface functionalizing agent. In certain embodiments, the surface functionalizing agent is an aminoorganosilane, such as 3-aminopropyltrimethoxysilane (APTMS), a maleimide, a thiol-maleimide, a glutaraldehyde, a dopamine, a hydroxypyrrolidine, a diisothiocyanate, a catechol, a polyester, or an aldehyde. In certain embodiments, the article is an implant or medical device.

Methods of improving and/or reducing foreign body reaction to an article are also provided. In certain embodiments, the method comprises coating at least one surface of the article with a surface functionalizing agent, wherein a hyaluronic acid binding peptide (HABP) is bound to the surface functionalizing agent to form a treated article and providing the treated article to a subject, wherein said providing comprises at least partially implanting the treated article within the subject.

Methods of decreasing inflammation and/or promoting wound healing in a subject are also provided. In certain embodiments, the method comprises providing to the subject a biomaterial comprising a hyaluronic acid binding peptide (HABP), wherein the HABP increases hyaluronic acid (HA) deposition in an area surrounding the biomaterial.

Methods of treating a dermal condition are also provided. In certain embodiments, the method comprises administering to a subject a composition comprising a hyaluronic acid binding peptide (HABP) bound to a surface functionalizing agent. In certain embodiments, the dermal condition comprises dermal condition comprises fine lines, wrinkles, scarring, dark spots, acne, xeroderma, cellulite, excess adipose, dermatitis, or volume defects.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE FIGURES

Various embodiments of this disclosure are described with reference to the figures. Reference to Figures, and any embodiments disclosed therein, does not limit the scope of the invention. Figures represented herein are not limitations to the various embodiments according to the disclosure and are presented as examples and non-limiting embodiments of the inventions disclosed herein.

The patent or application file contains at least one drawing executed in color. Copies of this patent or patent application publication with color drawing(s) will be provided by the Office upon request and payment of the necessary fee.

FIGS. 1A-D show surface functionalization via APTMS surface functionalization.

FIG. 1A shows silica glass surface with available silane groups. FIG. 1B shows 2% APTMS solution bound to the surface of the silica glass through a silane reaction, leaving an amine group available for binding. FIG. 1C shows HABP bound to the surface through attachment to the available amine group on the APTMS (bonding with the available carboxyl group. FIG. 1D shows Hyaluronic acid attracted to the peptide functionalized surface.

FIGS. 2A-C depict assessment of the cytocompatibility of the peptide treated surfaces using Live/Dead and PrestoBlue assays. Presto Blue assay is a measure of cellular metabolic activity. FIG. 2A is a color image of a cell culture: Calcein-AM (green) and Propidium Iodide (red) were used to stain for live and dead cells respectively. Cell viability of HABP was found to be 98.9%. FIG. 2B shows a graphical representation of the PrestoBlue surface treatment results indicated no significant difference between experimental groups. FIG. 2C shows a graphical representation of the cell viability results indicate no significant difference between HABP and control groups, indicating that HABP is cytocompatible.

FIGS. 3A-B depicts primary dermal fibroblasts seeded at 50.000 cells/154 mm² and incubated for 10 days on HABP and Scrambled HABP functionalized surfaces. APTMS surfaces were treated with 2% APTMS only and Untreated surfaces had no treatment other than PBS rinse. FIG. 3A shows Brightfield microscope images were taken on Days 1, 5, and 10 at 10× magnification. Decellularization occurred on Day 11. (Representative of average and standard deviation; N=3; scale bar represents 100 μm). FIG. 3B shows a graphical representation of the alignment values of primary dermal fibroblasts on day 5 of culture. No significant differences between days or treatment groups were noted. (Representative of average and standard deviation; N=6).

FIGS. 4A-B show Alcian Blue stained FDMs. FIG. 4A is a color cell culture stain: Blue staining indicates HA presence in cultures. HABP exhibited more specific staining than both control groups. Scale bar represents 100 μm. FIG. 4B is a graphical representation depicting the higher Alcian Blue intensity in HABP cultures to a significant degree. (Representative of average and standard deviation; N=3; p-value<0.05).

FIGS. 5A-G show Type I Collagen immunostaining results. FIG. 5A is a color cell culture stain: Fluorescent images of Col1 at 20× magnification. The scale bar represents 100 μm. FIG. 5B is a color image showing CT-Fire overlays of Col1 fibers from the above images, where different fiber characteristics are shown via different coloration. FIG. 5C is a graphical representation of the distribution of the number of Col1 fibers in each treatment group. FIG. 5D is a graphical representation of the distribution of Col1 fiber lengths in each treatment group. There is a notable difference between the APTMS group and the Untreated and HABP groups. FIG. 5E is a graphical representation of the measurement of fiber straightness. Fibers on Untreated and HABP-treated surfaces had a higher level of fiber straightness than APTMS. The closer the value is to one, the straighter individual fibers within the treatment groups are. FIG. 5F is a graphical representation of the Col1 fiber width comparison between treatment groups. A significant difference was found between the Untreated experimental group and the HABP and APTMS experimental groups, also indicating the APTMS effect on fiber dimensions. FIG. 5G is a graphical representation of the Col1 fiber number varies between experimental groups, but not to a significant degree. H. Measure of degree of fiber angles between experimental groups. (Representative of average and standard deviation; N=9); * p-value<0.01; ** p-value<0.001; all units are in microns).

FIGS. 6A-D shows a cell culture stain of actin (green) and α-SMA (red) staining of dermal fibroblasts after 48 hours in culture. FIG. 6A shows a color image of a cell culture stain of actin (green) and α-SMA (red) staining of dermal fibroblasts after 48 hours in culture. There were fewer cells noted in untreated cultures, with less actin signal, indicating that cells do not attach as well to the untreated surface. FIG. 6B is a graph of Actin and α-SMA fluorescent signal. FIG. 6C is a graph of percent area covered by F-actin in each culture, with the untreated control having significantly less coverage. FIG. 6D is a graph of the mean intensity from the fluorescent images. HABP had a significantly lower mean intensity than the two controls. (Representative of average and standard deviation; N=3 *** p-value<0.001. **** p-value<0.0001; scale bar represents 100 μm).

FIGS. 7A-C show Alcian Blue stained FDMs. FIG. 7A Blue staining indicates HA presence in cultures. HABP exhibited more specific staining than the control. FIG. 7B Graph depicting the higher Alcian Blue intensity in HABP cultures to a significant degree. (Representative of average and standard deviation; N=3; p-value<0.05). FIG. 7C shows a color photograph of the HABP plate and Control plate including a zoomed-in portion of each, showing the HABP exhibited more specific staining than the control.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. The definitions are provided to aid in describing particular embodiments and are not intended to limit the claimed disclosure. Many methods and materials similar, modified, or equivalent to those described herein can be used in the practice of the embodiments without undue experimentation, but the preferred materials and methods are described herein. In describing and claiming the embodiments, the following terminology will be used in accordance with the definitions set out below.

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

As used herein, the term “and/or”, e.g., “X and/or Y” shall be understood to mean either “X and Y” or “X or Y” and shall be taken to provide explicit support for both meanings or for either meaning, e.g., A and/or B includes the options i) A, ii) B or iii) A and B.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

The term “about,” as used herein, refers to variation in the numerical quantity that can occur, for example, through typical measuring techniques and equipment, with respect to any quantifiable variable, including, but not limited to, concentration, count, log, mass, pH, time, temperature, viability, and volume. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

As used herein, the terms “include,” “includes,” and “including” are to be construed as at least having the features to which they refer while not excluding any additional unspecified features.

As used herein, “pharmaceutically acceptable” refers to compounds, materials, compositions, and/or dosage forms which are suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. A “pharmaceutically acceptable excipient” includes any and all carriers, solvents, growth media, dispersion media, coatings, adjuvants, fillers, buffers, stabilizers, lubricants, stabilizing agents, diluents, preservatives, inactivating agents, antimicrobial, antibacterial and antifungal agents, isotonic agents, adsorption delaying agents, and the like. Such ingredients include those that are safe and appropriate for use in medical or pharmaceutical applications.

The term “subject” as used herein refers to any living being that would benefit from the compositions and methods described herein. “Subject” can be used interchangeably with “patient”. For example, the subject or patient may be an animal, including a human, avian, bovine, canine, equine, feline, hircine, lupine, murine, ovine, and porcine animal. Subjects may be domesticated animals such as cats, dogs, rabbits, guinea pigs, ferrets, hamsters, mice, gerbils, horses, cows, goats, sheep, donkeys, pigs, and the like. In certain embodiments, the subject or patient is a human.

The term “actives” or “percent actives” or “percent by weight actives” or “actives concentration” are used interchangeably herein and refers to the concentration of those ingredients involved in cleaning expressed as a percentage minus inert ingredients such as water or salts. It is also sometimes indicated by a percentage in parentheses, for example, “chemical (10%).”

The term “weight percent,” “wt. %,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt. %,” etc.

The methods and compositions may comprise, consist essentially of, or consist of the components and ingredients as well as other ingredients described herein. As used herein, “consisting essentially of” means that the methods and compositions may include additional steps, components or ingredients, but only if the additional steps, components or ingredients do not materially alter the basic and novel characteristics of the claimed methods and compositions.

### HABP Treated Articles

The present disclosure provides articles treated with hyaluronic acid binding peptide (HABP). Beneficially, these treated articles can reduce or diminish fibrosis and other negative effects of foreign body reaction against the article.

In certain embodiments, the article is an implant, medical device, or prosthetic. In certain embodiments, the article is at least partially implanted within the subject. As used herein, “implant” refers to a device manufactured to replace, support, or enhance an existing biological structure. The implant may be anp orthopedic implant, such as, for example, a pin, rod, screw, plate, or artificial joint. The implant may replace or support a joint, such as a hip, knee, elbow; ankle, shoulder, or wrist. The implant may be a bone or dental implant. The implant may be a surgical or wound mesh. The implant may be a sensory or neurological implant, for example, a cochlear implant, intraocular lens, tympanostomy tube, neurostimulator, or the like. The implant may be a cardiovascular implant, for example, an artificial heart, an artificial heart valve, an implantable cardioverter-defibrillator, a pacemaker, a coronary stent, or the like. The implant may be a contraceptive implant, such as a copper- or hormone-base intrauterine device. The implant may be a cosmetic article or a cosmetic implant, such as a breast implant, nose prosthesis, ocular prosthesis, injectable filler, or the like. The implant may also be an implant used to treat various conditions and/or organ dysfunction, for example, an implantable gastric stimulator, diaphragmatic/phrenic nerve stimulator, artificial urinary sphincter, penile implant, catheter, or the like.

### Surface Functionalizing Agent

Articles of the present disclosure comprise a coating of a surface functionalizing agent on a surface of the article. The surface functionalizing agent alters the surface and changes how the surface of the article interacts with the surrounding environment. Surface functionalizing agents can be used to bind a bioactive component, such as HABP, to the surface of the article. Said binding can also be referred to as “surface immobilization” and includes, for example, electrostatic interactions, covalent binding, and affinity interactions (e.g., hydrogen bonds, van der Waals forces, London dispersion forces, etc.).

Surface immobilization provides a higher level of control over surface interactions. Non-specific reactions are typical with the introduction of an implant to the body; immobilizing a specific bioactive molecule, like HABP, on the surface allows for inhibition of the non-specific reactions in favor of the desired reaction. Surface modification should be done for the purpose of combining properties from the substrate with specific intermolecular interactions. There are two important steps in surface modification with a surface functionalizing agent: surface activation and graft polymerization. Surface activation often involves addition of reactive chemical moiety onto the surface, such as —OH, —COOH, —NH₂ and —SH groups that have ability to form bonds with biological molecules or any polymer carrying reactive groups. Graft polymerization is defined as the molecular assembly at the surface between the reactive groups and the graft material.

In certain embodiments, the surface activating agent is an organosilane. Organosilanes have a general structure of R—Si(OX)₃, wherein “R” can be a number of different non-hydrolysable organic moieties, and versatile, bi-functional compounds. There are several different types of organosilanes including trialkoxysilanes, dimethylsilanols, and aminoorganosilanes. Organosilanes are most often used as coupling agents because they are capable of forming a stable bridge between a hydrophilic surface and an organic molecule. Additionally, they are highly chemoselective based on their R group, and therefore establish the specific intermolecular interactions desired in surface modification procedures. These specific interactions are bolstered by the secondary bonding (van der Waals forces, hydrogen bonding, dipole-dipole interactions) promoted by the organosilanes. Organosilanes not only bond to the surface through covalent bonding, but they also bond to each other to form self-assembled monolayers (SAMs). The “R” and “X” groups on the molecule provide variability to choose from when determining desired surface properties.

In certain embodiments, the surface functionalizing agent is an aminoorganosilane. Aminoorganosilanes are organosilanes with an amine group as the “R” component of the molecule. In certain embodiments, the aminoorganosilane is 3-aminopropyltrimethoxysilane (APTMS). APTMS is ideal due in part to its extended electrostatic potential. APTMS consists of an amino group attached to an alkyl chain bound to a silane with three methoxy groups. Both the amino and methoxy arms of the APTMS will compete for the hydroxyl groups that are present on many biomaterial surfaces, like glass. APTMS has the following structure:

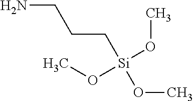

Surface hydroxylation is the introduction of hydroxyl groups to a material surface; this process is carried out upon the introduction of APTMS to a material surface. APTMS becomes bound to a material surface through chemisorption, which is the process during which chemical specificity is attained and there are changes in the electronic state as a result of the surface reaction. When APTMS is introduced to a surface as a fluid, they interact with the surface and form layers through denominated adsorption. The SAM formation is completed in four steps: hydrolysis of the silane group, hydrogen bonding to the surface hydroxyl groups, condensation with neighboring silane molecules (where the APTMS molecules form bonds with each other), and condensation with the surface. The final step in this reaction is a polymerization step where siloxanes are formed, meaning the silane is covalently bonded to the surface. Dry solvents, like alcohols, are used to control silane layer formation and to prevent the formation of disorganized layers. Dry solvents control the reaction so that the hydrolysis only occurs at the silica surface. In the case of APTMS, acetone, ethanol, and isopropyl alcohol are ideal solvents. This controlled reaction leaves the amine group free, creating a functionalized surface with specific surface chemistry.

When functionalizing a surface, it is crucial that the procedure be reproducible so that similar results can be attained consistently. The reactivity and density of reactive receptor sites and the APTMS layer morphology, stability, and thickness should all be considered. The surface should be functionalized with groups that are specific to the bioactive species of interest. In the case of APTMS, the bioactive molecule should be able to react with the available amine groups. It is important that the APTMS layer be organized so that amine groups are accessible. Optimization of reactive site density is more important than maximizing them to avoid overcrowding. In certain embodiments, the surface is covered homogenously so that there is an even density of reaction sites across the material. This optimization also contributes to more even layers. Exposure to water is extremely harmful to an even, stable layer formation because water is one of the biggest competing agents that can cause a reduction in bond strength. If polymerization of APTMS occurs, it causes the formation of multiple layers, which contributes to the overall thickness of the APTMS layer. While a thick layer is more ideal for industrial applications, in biomedical applications, a monolayer is both more stable and reproducible, and therefore is more ideal for certain applications.

The surface can be comprised of any material that is compatible with the surface functionalizing agent and intended use of the article. In certain embodiments, the surface is comprised of metal, such as, for example, stainless steel, titanium alloy, cobalt chrome alloy, tantalum, or the like. In certain embodiments, the surface is comprised of glass, such as silica glass. In certain embodiments, the surface is comprised of silicon. In certain embodiments, the surface is comprised of a polymer, ceramic, glass, or plastic. In some embodiments, the surface can be coated. Surface coatings can include, but are not limited to, bifunctional cross-linkers, surface functional agents, self-assembling materials, organosilanes, click chemistry, click chemistry compatible linkers, cross-linking amino acids (such as dopamine), plasma, infrared and/or UV-assisted surface coatings, or mixtures thereof.

In certain embodiments, the surface functionalizing agent comprises a polyester, an acrylamide, biological polymer (including, but not limited to, alginate, HA, elastin, fibrinogen/fibrin, collagen, carotin, silk, silk fibronin, sericin, and mixtures thereof) and any degradable and non-degradable biologically compatible synthetic polymers (including, but not limited to, polydioxanone, polyhydroxy butyrate, polyhydroxyvalarate, polyanhydrides, polyorthoesters, polycyanoacrylates, polyphosphazines, polyglycolic acid, polylactic acid, and mixtures thereof).

In certain embodiments, the surface functionalizing agent comprises a maleimide, a thiol-maleimide, a glutaraldehyde, a dopamine, a hydroxypyrrolidine, a diisothiocyanate, a catechol, or an aldehyde. In some embodiments, adipic hydrazide is reacted with an aldehyde to form a covalent linkage.

### Hyaluronic Acid Binding Peptide

Hyaluronic Acid (HA) is a non-sulfated, linear, and hydrophilic glycosaminoglycan with repeating disaccharides of (β, 1-4)-gluronic acid and (β, 1-3)-N-acetyl glucosamine. As a component of the extracellular matrix (ECM), HA plays an important role in cell migration, tissue structure, and cell proliferation processes. It also aids in hydration and fluid viscosity, which is why HA is most often found in hydrated tissues like the skin, vitreous humor in the eye, and synovial fluid. HA, unlike other glycosaminoglycans (GAGs) is synthesized by three separate single transmembrane HA synthases (HAS). HA can be both high and low molecular weight; native HA is most often high molecular weight (1-10 million Da). HMW HA is synthesized by HAS1 and HAS2, while LMW HA is synthesized by HAS3, which has been found to be more active than the other two synthases. HWM HA is typically larger than 500 disaccharide units, while LMW HA is anything lower. HA function is regulated by the two signal-transducing cell surface HA-receptors: CD44 and receptor for hyaluronan-mediated motility (RHAMM), which are responsible for HA-mediated cell interactions and HA-mediated motility respectively.

During inflammation, HA interacts with other ECM components and HA chains can become organized into ECM structures through binding protein associations. HA cross-linking has been shown as a mechanism for inflammation regulation, which makes HA a viable therapeutic strategy in regard to preventing fibrotic capsule formation (FCF). CD44 and RHAMM mediate the physiological and pathological functions of HA, and HA attachment to these receptors contributes to activation of intracellular signaling pathways and gene expression induction related to inflammation and wound healing among other things, giving HA a role in regulating these processes. HA, being a biological molecule existing within and having a role in regulating the wound healing process, is an ideal molecule for use in preventing FCF.

However, while HMW HA aids in regulating inflammation and wound healing, LMW is thought to induce inflammatory responses. As inflammation progresses, HMW HA is broken down into fragments, which results in higher concentrations of LMW HA and contributes to prolonging inflammation. Prolonged inflammation can eventually lead to fibrosis as well. HA is also an expensive biological molecule, which raises the question of whether there are alternative strategies to increase HA concentration without introducing HA itself as the therapeutic strategy, such as inducing cells to produce their own HA.

As mentioned above, HA is prone to cross-linking, which is part of its use in regulating inflammation. Many proteins found in the ECM are capable of binding to HA through a common domain called a Link module, which is a component involved in ligand binding. The link modules are tandem repeat short peptide sequences can be found in ECM proteins like aggrecan and versican, allowing them to form bonds with HA.

Link modules interact with HA to form stabilized protein complexes and function in contributing to the structural integrity of many tissues. The link module is made up of around 100 amino acids with four cysteines. The cysteines are disulfide-bonded as Cys1-Cys4 and Cys2-Cys3, contributing to a three-dimensional (3D) structure. The organization of the link module result in a characteristic fold, which serves to identify members of the Link module superfamily. CD44 has a single link module consisting of approximately 160 amino acids. HABPs are isolated from the RHAMM molecular sequence (Table 1), and like several ECM proteins, are capable of binding to HA. HABPs are capable of altering the HA-binding capacity of HA-receptors, allowing for more control of disease progression, including the progression of fibrosis. HABP is an ideal molecule to use for encouragement for cells to deposit their own HA because not only has it been shown to be capable of doing so, it can also be isolated and bound to functionalized surfaces. This ability to bind to functionalized surfaces is ideal for preventing FCF around implants.

TABLE 1
Synthesized HABP sequences.

| Peptide | Isolated | |
|---|---|---|
| Sequence | From | SEQ ID NO |
| GAHWQFNALTVR | RHAMM | SEQ ID NO: 1 |
| STMMSRSHKTRS | RHAMM | SEQ ID NO: 2 |
| HHV | | |
| YKQKIKHVVKLK | RHAMM | SEQ ID NO: 3 |
| RYPISRPRKRC | RHAMM | SEQ ID NO: 4 |
| LKQKIKHVVKLK | RHAMM | SEQ ID NO: 5 |
| VVVKLRSQLVKR | | |
| KQN | | |

In certain embodiments, the HABP comprises the amino acid sequence of any one of SEQ ID NOs: 1, 2, 3, 4, or 5. In certain embodiments, the HABP comprises the amino acid sequence of SEQ ID NO: 1.

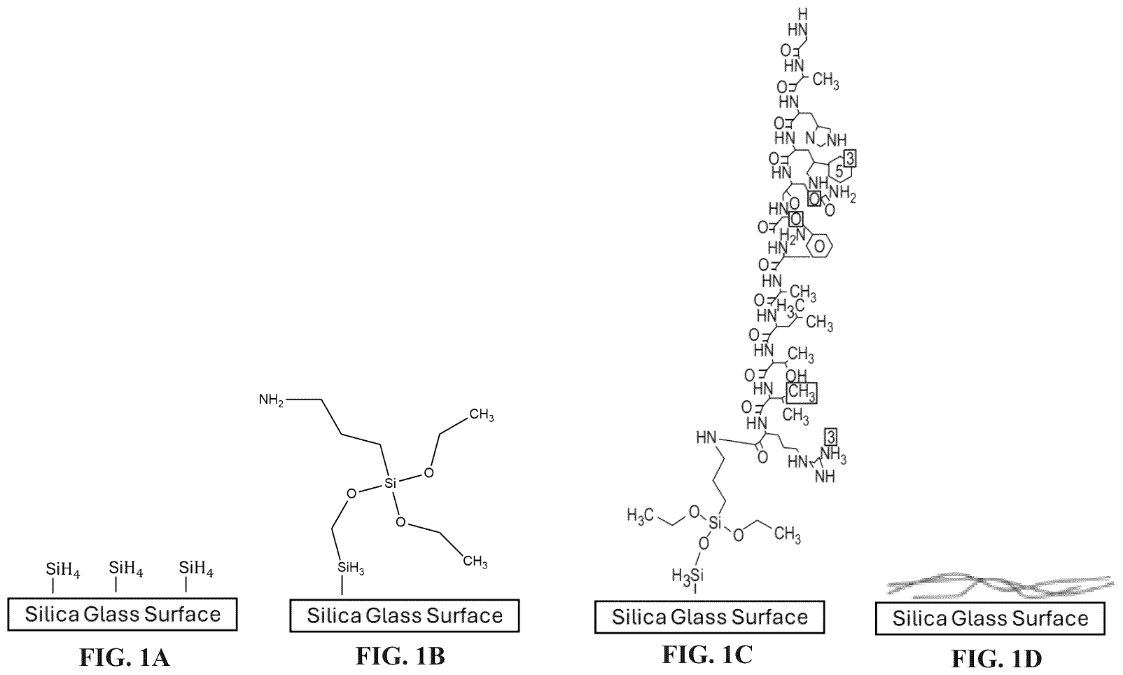

In certain embodiments, the HABP binds to the surface functionalizing agent that is coating the article. This is shown in FIG. 1. As shown in the figure, the surface functionalizing agent does not bind HA directly, but instead binds HABP which attracts HA deposition and induces cellular changes in surrounding fibroblasts.

Articles of the present disclosure may further be coated with additional components. In some embodiments, the article is also coated with a compound that prevents degradation of HA, such as aurothiomalate, indomethacin, propylene glycol, heparin, dextran sulphate, fucoidan, and/or carboxymethyl cellulose. In some embodiments, the article is also coated with or contains an antimicrobial, antibiotic, antiviral, or the like.

The HABP can also be provided in combination with any number of other beneficial peptides, including, but not limited to, collagen mimetic peptides, thymosin beta 5, imechano growth factor (MGF) peptide, IGF-1, CJC-1295, ipamorelin, and mixtures thereof. In certain embodiments, more than one peptide (i.e. HABP and at least one additional peptide) binds to the surface functionalizing agent. In certain embodiments, the additional peptide(s) enhances the retention of HABP.

### Methods of Reducing Foreign Body Reaction

Methods of improving and/or reducing foreign body reaction to an article are also provided. In certain embodiments, the method comprises coating at least one surface of the article with a surface functionalizing agent as described herein.

A HABP is bound to the surface functionalizing agent to form a treated article. The treated article can be provided to a subject. Said providing step will vary depending on the type of article, its intended purpose, and the anatomical location in which the article will be placed. In certain embodiments, the providing step comprises at least partially implanting the treated article within the body of the subject. In certain embodiments, the article is fully or completely implanted within the body of the subject. In certain embodiments, at least a portion of the article remains outside of the subject's body. For example, in the case of artificial heart valves, artificial joints, and the like, said providing step could comprise implanting the article fully within the body of the subject. In the case of cochlear implants, catheters, and the like, there may be a portion of the article that remains outside of the subject's body and the providing step could comprise only partial implantation of the article.

The article may be partially or completely coated with the surface functionalizing agent and bound HABP. In certain embodiments, for example, in cases where only a portion of the article will be implanted within the subject's body, it may be desirable to only coat the portion of the article that will be implanted. In other embodiments, it may be desirable to coat the entire article.

The articles and methods described herein can be used for a variety of clinical applications, including for example: as a dermal implant for cosmetic applications; for visco-supplementation in joints; as a medical device to augment bone growth; as an implant in spinal fusion surgery; as a surgical sling, mesh, or patch; as an implant for the treatment of periodontal disease (e.g., as a dental implant); as a skin graft (e.g., for the development of artificial skin); as a corneal shield; as a tissue bulking agent for the treatment of urinary incontinence, fecal incontinence, or gastro-esophageal reflux; as a surgical adhesion barrier; or as a glaucoma drainage device.

While not wishing to be bound by theory, it is believed that treated articles of the present disclosure can reduce, diminish, or lessen the occurrence or severity of foreign body reaction as compared to untreated articles by not only increasing HA deposition in an area surrounding the treated article, but also by inducing cellular changes within surrounding fibroblasts. In certain embodiments, fibroblast cells in an area surrounding the treated article exhibit altered behavior as compared to fibroblast cells in an area surrounding an untreated article. In some embodiments, said altered behavior comprises increased collagen fiber organization, decreased collagen fiber length, and/or reduced collagen fiber formation, all of which are associated with reduced fibrosis, fibrotic capsule formation, and foreign body reaction.

### Additional Methods

Other methods of using HABP and/or providing HABP to a subject are also provided. Methods of decreasing inflammation and/or promoting wound healing in a subject are provided. In certain embodiments, the method comprises providing to the subject a biomaterial comprising a HABP bound to a surface functionalizing agent.

The term “biomaterial” as used herein, refers generally to a substance or article that has been engineered to interact with biological systems for a medical purpose and/or that have a biological use. Biomaterials can be in a variety of forms. In some embodiments, the biomaterial is flowable. For example, the biomaterial may be a liquid, gel, hydrogel, cream, or ointment. In some embodiments, the biomaterial can be a wound dressing, wound mesh, surgical mesh, nanofiber, microfiber, sponge, suture, or the like. In certain embodiments, the biomaterial is biodegradable. In certain embodiments, the biomaterial is not biodegradable.

Biomaterials can further comprise additional functional and/or adjuvant components. Preferably, additional components will be pharmaceutically acceptable and compatible with the HABP and surface functionalizing agent. Additional components may comprise a carrier, adjuvant, diluent, buffer, stabilizer, preservative, lubricant, and/or the like. Additional components may be selected based on a number of factors, including intended use of the biomaterial, the disease, disorder or condition being treated with the biomaterial, the species of subject, the age, size and general condition of the subject, and the formulation of the biomaterial. Additional components may comprise, for example, therapeutic materials, bioactive molecules, biominerals, hydroxyapatite, bioactive calcium phosphate, gutta-percha, latex, antibiotics, antibacterial agents, anti-viral agents, cariostatics, gelling agents, anti-inflammatory agents, anti-oxidants, adhesives, crystal adjustors, viscosity modifiers, preservatives, plasticizers, pore forming agents, fibers and meshes, metals, oxides, bone chips, bone crystals, organic and mineral fraction of bones and teeth, polymeric resins, resorbable and biodegradable and nonresorbable and naturally-occurring and synthetic polymers and copolymers, biologic factors, bioactive substances and cells, drugs, proteins, hormones, enzymes, antigens, immunogens, cytotoxins, neurotransmitters, interferons, interleukins, chemokines, cytokines, extracellular matrix components, synthetic peptide containing powder, adhesion molecules, ligands and peptides, osteoinductive factors, nano-particles, nanotubes, nanofibers, and combinations thereof.

The method comprises providing the biomaterial to the subject. Said providing step will vary depending on the type and form of the biomaterial, its intended purpose, and the anatomical location in which the biomaterial is intended to be introduced. In certain embodiments, the providing step comprises topical application of the biomaterial. In certain embodiments, the providing step can comprise injecting the biomaterial into the skin, tissue, or joint of the subject.

Biomaterials of the present disclosure can decrease inflammation and promote wound healing, in part by preventing or reducing the occurrence or severity of foreign body reaction. While not wishing to be bound by theory, it is believed that biomaterials of the present disclosure not only increase HA deposition in an area surrounding the treated article, but also induce cellular changes within surrounding fibroblasts. In certain embodiments, fibroblast cells in an area surrounding the biomaterial exhibit altered behavior as compared to fibroblast cells not in an area surrounding the biomaterial. In some embodiments, said altered behavior comprises increased collagen fiber organization, decreased collagen fiber length, and/or reduced collagen fiber formation, all of which are associated with reduced fibrosis, fibrotic capsule formation, and foreign body reaction.

In some embodiments, the HABP can be provided in a composition as a treatment directly to a patient, including via micro-needling, microabrasion therapy, mesotherapy, roller therapy, and other cosmetic and dermal applications where the HABP is provided to a patient's skin. These embodiments may comprise a mesotherapy application, intradermal application, or subdermal application.

Methods of treating a dermal condition are also provided. The method can comprise administering to a subject a composition comprising a hyaluronic acid binding peptide (HABP) bound to a surface functionalizing agent. The dermal condition may be, for example, fine lines, wrinkles, scarring, dark spots, acne, xeroderma, cellulite, excess adipose, dermatitis, or volume defects. The HABP compositions can be useful for treating and preventing cutaneous signs of aging or external factors such as stress, air pollution, tobacco, or prolonged exposure to ultraviolet exposure. The HABP compositions can further improve the surface appearance, viscoelastic, and biomechanical properties of skin and can be useful in filling volume defects of the skin.

Administration can be subdermal, intradermal, or subcutaneous injection into the skin of a subject. Any body area in need may be treated, including, for example, facial, hand, neck, or abdominal skin.

Compositions and biomaterials of the present disclosure can contain additional functional and/or nonfunctional ingredients, including, for example, anti-itch, anti-cellulite, anti-scarring, and anti-inflammatory agents, anesthetics, anti-irritants, vasoconstrictors, vasodilators, as well as agents to prevent/stop bleeding, and improve/remove pigmentation, moisturizers, desquamating agents, tensioning agents, anti-acne agents. Anti-itch agents can include methyl sulphonyl methane, sodium bicarbonate, calamine, allantoin, kaolin, peppermint, tea tree oil, camphor, menthol, hydrocortisone and combinations thereof. Anti-cellulite agents can include forskolin, xanthine compounds such as, but not limited to, caffeine, theophylline, theobromine, and aminophylline, and combinations thereof. Anesthetic agents can include lidocaine, benzocaine, butamben, dibucaine, oxy buprocaine, pramoxine, proparacaine, proxymetacaine, tetracaine, and combinations thereof. Anti-scarring agents can include IFN-gamma., fluorouracil, poly(lactic-co-glycolic acid), methylated polyethylene glycol, polylactic acid, polyethylene glycol and combinations thereof. Anti-inflammatory agents can include dexamethasone, prednisolone, corticosterone, budesonide, estrogen, sulfasalazine, mesalamine, cetirizine, diphenhydramine, antipyrine, methyl salicylate, loratadine, and derivatives and combinations thereof. Additionally, active agents such as epinephrine, thymidine, cytidine, uridine, antiypyrin, aminocaproic acid, tranexamic acid, eucalyptol, allantoin, glycerin, and sodium selenite, can be included. The disclosed HABP compositions can further comprise degradation inhibitors. Degradation inhibitors, include but are not limited to, glycosaminoglycans (e.g., heparin, heparin sulfate, dermatan sulfate, chondroitin sulfate, o-sulfated HA, linamarin, glucosamine, and amygdalin), antioxidants (e.g. ascorbic acid, melatonin, vitamin C, vitamin E, sodium selenite, glutathion, retinoic acid, coenzyme, beta-carotene, allopurinol, mannitol, caffeic acid, caffeine, polyphenol, theobromine, catechin), proteins (e.g., serum hyaluronidase inhibitor), and fatty acids (e.g. saturated C10 to C22 fatty acids), vitamin B and complex, and combinations thereof as noted. In certain embodiments, the additional ingredient can be an antioxidant. In certain embodiments, the antioxidant comprises a vitamin C such as ascorbyl-2-glucoside and/or a vitamin E such as d-alpha-tocopheryl polyethylene glycol 1000 succinate (TPGS). Anti-irritants can include thymol, bisabolol. Healing agents can include allantoin, eucalyptol, chitosane, cytidine, thimidine, uridine, lanoline. Anti-bleeding agents can include epinephrine, norepinephrine, phenylephrine, synephrine, naphazoline, aminocaproic acid, tranexamic acid, ethamsylate, vitamin K. Collagen promoters can include retinol or additional peptide sequences, including, but not limited to, collagen mimetic peptides, thymosin beta 5, imechano growth factor (MGF) peptide. IGF-1. CJC-1295, ipamorelin, and mixtures thereof.

Other optional ingredients include, for example, emulsifying agents, wetting agents, sweetening or flavoring agents, tonicity adjusters, preservatives, buffers, antioxidants, and flavonoids. Tonicity adjustors useful in embodiments of the present disclosure include, but are not limited to, salts such as sodium acetate, sodium chloride, potassium chloride, mannitol or glycerin and other pharmaceutically/cosmetically acceptable tonicity adjusters. Preservatives can include, without limitation, benzalkonium chloride, chlorobutanol, thimerosal, phenyl mercuric acetate, and phenyl mercuric nitrate. Various buffers and means for adjusting pH can be used, including but not limited to, acetate buffers, citrate buffers, phosphate buffers and borate buffers. Similarly, antioxidants can include, for example, sodium metabisulfite, sodium thiosulfate, acetylcysteine, butylated hydroxyanisole and butylated hydroxytoluene. Flavonoids are compounds found in plants that are well known to have diverse beneficial biochemical and antioxidant effects. Subcategories of flavonoids include: flavones, flavonols, flavanones and flavanonols. Examples of flavonoids include: luteolin, apigenin, tangeritin, quercetin, kaempferol, myricetin, fisetin, isorhamnetin, pachypodol, rhamnazin, hesperetin, naringenin, eriodictyol, homoeriodictyol, taxifolin, dihydroquercetin, dihydrokaempferol, tannic acid, tannins, condensed tannins, and hydrolysable tannins. The pH of the disclosed biomaterials and compositions can be about 5.0 to about 8.0, or about 6.5 to about 7.5. In certain embodiments, the pH of the formulation is about 7.0 to about 7.4 or about 7.1 to about 7.3.

The disclosed compositions are also well-suited for mesotherapy. Mesotherapy is a non-surgical cosmetic treatment technique involving intradermal and/or subcutaneous injection of an agent (micronutrients, vitamins, mineral salts, etc). The compositions are administered in the form of small multiple droplets into the epidermis, dermo-epidermal junction, and/or the dermis.

The HABP compositions can be injected utilizing needles with a diameter of about 0.26 to about 0.4 mm and a length ranging from about 4 to about 14 mm. Alternately, the needles can be 21 to 32 g and have a length of about 4 mm to about 70 mm. Preferably, the needle is a single-use needle. The needle can be combined with a syringe, catheter, and/or a pistol (for example, a hydropneumatic-compression pistol).

The HABP compositions can be administered once or over several sessions with the subject spaced apart by a few days, or weeks. For instance, the subject can be administered a formulation every 1, 2, 3, 4, 5, 6, 7, days or every 1, 2, 3, or 4, weeks. The administration can be on a monthly or bi-monthly basis. Further, the formulation can be administered every 3, 6, 9, or 12 months.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### Example Embodiments

The inventions are defined in the claims. However, below is a non-exhaustive list of non-limiting embodiments in numbered format. Any one or more of the features of these embodiments may be combined with any one or more features of another example, embodiment, or aspect described herein. Accordingly, the following numbered clauses form part of the present disclosure but do not form part of the claims:

Clause 1. A method of decreasing inflammation and/or promoting wound healing in a patient, comprising: providing to the patient a biomaterial comprising a hyaluronic acid binding peptide (HABP), wherein the HABP increases hyaluronic acid (HA) deposition in an area surrounding the biomaterial.

Clause 2. The method of clause 1, wherein the HABP is bound to a surface functionalizing agent.

Clause 3. The method of clause 2, wherein the surface functionalizing agent is an aminoorganosilane, a maleimide, a thiol-maleimide, a glutaraldehyde, a dopamine, a hydroxypyrrolidine, a diisothiocyanate, a catechol, a polyester, and/or an aldehyde.

Clause 4. The method of clause 2, wherein the surface functionalizing agent is an aminoorganosilane.

Clause 5. The method of clause 4, wherein the aminoorganosilane is 3-aminopropyltrimethoxysilane (APTMS).

Clause 6. The method of clause 1, wherein the biomaterial is a liquid, gel, hydrogel, wound dressing, wound mesh, surgical mesh, nanofiber, and/or suture.

Clause 7. The method of clause 1, wherein the HABP comprises the amino acid sequence of any one of SEQ ID NOs: 1, 2, 3, 4, or 5.

Clause 8. The method of clause 1, wherein the HABP comprises the amino acid sequence of SEQ ID NO: 1.

Clause 9. The method of clause 1, wherein said providing step comprises injecting the biomaterial into the skin or a tissue of the patient.

Clause 10. The method of clause 1, wherein said providing step comprises topical application of the biomaterial.

Clause 11. The method of clause 2, wherein the biomaterial is an implant or medical device.

Clause 12. The method of clause 11, wherein at least one surface of the implant or medical device is coated with the surface functionalizing agent bound to the HABP.

Clause 13. The method of clause 1, wherein the biomaterial improves and/or reduces a foreign body reaction.

Clause 14. The method of clause 13, wherein said foreign body reaction comprises fibrotic capsule formation.

Clause 15. The method of clause 1, wherein fibroblast cells in an area surrounding the biomaterial exhibit increased collagen fiber organization, decreased collagen fiber length, and/or reduced collagen fiber formation.

Clause 16. An article comprising: a coating of a surface functionalizing agent on a surface of the article, wherein a hyaluronic acid binding peptide (HABP) is bound to the surface functionalizing agent; wherein the surface functionalizing agent is selected from the group consisting of an aminoorganosilane, a maleimide, a thiol-maleimide, a glutaraldehyde, a dopamine, a hydroxypyrrolidine, a diisothiocyanate, a catechol, a polyester, and an aldehyde.

Clause 17. The article of clause 16, wherein the surface functionalizing agent is an aminoorganosilane.

Clause 18. The article of clause 17, wherein the aminoorganosilane is 3-aminopropyltrimethoxysilane (APTMS).

Clause 19. The article of clause 16, wherein said article is an implant or medical device.

Clause 20. The article of clause 16, wherein the HABP comprises the amino acid sequence of any one of SEQ ID NOs: 1, 2, 3, 4, or 5.

Clause 21. The article of clause 16, wherein the HABP comprises the amino acid sequence of SEQ ID NO: 1.

Clause 22. The article of clause 16, wherein the surface is comprised of silica glass or silicon.

Clause 23. A method of improving and/or reducing foreign body reaction to an article, comprising: coating at least one surface of the article with a surface functionalizing agent, wherein a hyaluronic acid binding peptide (HABP) is bound to the surface functionalizing agent to form a treated article; and providing the treated article to a patient, wherein said providing comprises at least partially implanting the treated article within the patient.

Clause 24. The method of clause 23, wherein the surface functionalizing agent is an aminoorganosilane, a maleimide, a thiol-maleimide, a glutaraldehyde, a dopamine, a hydroxypyrrolidine, a diisothiocyanate, a catechol, a polyester, and/or an aldehyde.

Clause 25. The method of clause 24, wherein the aminoorganosilane is 3-aminopropyltrimethoxysilane (APTMS).

Clause 26. The method of clause 23, wherein the treated article is an implant or medical device.

Clause 27. The method of clause 23, wherein the HABP comprises the amino acid sequence of any one of SEQ ID NOs: 1, 2, 3, 4, or 5.

Clause 28. The method of clause 23, wherein the HABP comprises the amino acid sequence of SEQ ID NO: 1.

Clause 29. The method of clause 23, wherein the surface is comprised of silica glass or silicon.

Clause 30. The method of clause 23, wherein the HABP increases hyaluronic acid (HA) deposition in an area surrounding the treated article.

Clause 31. The method of clause 23, wherein fibroblast cells in an area surrounding the treated article exhibit altered behavior as compared to fibroblast cells in an area surrounding an untreated article.

Clause 32. The method of clause 31, wherein said altered behavior comprises increased collagen fiber organization, decreased collagen fiber length, and/or reduced collagen fiber formation.

Clause 33. The method of clause 23, wherein said foreign body reaction comprises fibrotic capsule formation.

Clause 34. A method of treating a dermal condition, comprising: administering to a patient a composition comprising a hyaluronic acid binding peptide (HABP) bound to a surface functionalizing agent, wherein the surface functionalizing agent is selected from the group consisting of an aminoorganosilane, a maleimide, a thiol-maleimide, a glutaraldehyde, a dopamine, a hydroxypyrrolidine, a diisothiocyanate, a catechol, a polyester, and an aldehyde.

Clause 35. The method of clause 34, wherein the surface functionalizing agent is an aminoorganosilane.

Clause 36. The method of clause 35, wherein the aminoorganosilane is 3-aminopropyltrimethoxysilane (APTMS).

Clause 37. The method of clause 34, wherein the HABP comprises the amino acid sequence of any one of SEQ ID NOs: 1, 2, 3, 4, or 5.

Clause 38. The method of clause 34, wherein the HABP comprises the amino acid sequence of SEQ ID NO: 1.

Clause 39. The method of clause 34, wherein said administering comprises subdermal, subcutaneous, or intradermal injection.

Clause 40. The method of clause 34, wherein said dermal condition comprises fine lines, wrinkles, scarring, dark spots, acne, xeroderma, cellulite, excess adipose, dermatitis, or volume defects.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1. Immobilization of HABP with Organosilanes as a Method of Preventing Fibrotic Capsule Formation

Functionalizing surfaces using organosilanes is a viable method for the immobilization of bioactive molecules. In the case of HABP, using an aminoorganosilane as a surface functionalizing agent is a novel approach to binding peptides to a surface. Functionalizing surfaces with HABP will increase the concentration of HA on the surface and contribute to reducing FCF through impacting fibroblast ECM deposition.

FIG. 1 depicts the steps taken during the HABP chemisorption onto a glass surface. Briefly, the glass surfaces are cleaned under sterile conditions followed by 3-aminopropyltrimethoxysilane (APTMS) exposure to create a self-assembled monolayer of polymerized APTMS. The polymerized APTMS expose free amine groups on the surface. The free amines are further allowed to react with HABP peptide sequence (GAHWQFNALTVR; SEQ ID NO: 1) under pH 7.0 to form amide bonds between the free NH₂ groups of the surface and the COOH groups of the C-terminus of peptides. Following the completion of the reaction, surfaces are washed and further processed immediately for experimentation. The peptide binding confirmation and the further characterization experiments were completed to characterize surfaces.

### Materials and Methods

The following materials were used: DI H2O, hydrochloric acid (Fischer Scientific), isopropyl alcohol (Sigma-Aldrich), ethanol (Sigma-Aldrich), 3-Aminopropyltrimethoxysilane (APTMS) (Sigma-Aldrich), phosphate buffered saline (PBS) (gibco), hyaluronic acid binding peptide (Pep1(GR-12). P210608-LL061233), scrambled hyaluronic acid binding peptide (Pep1(GR-12). P210630-LL061233), phosphate buffered saline (gibco), normal human adult primary dermal fibroblasts (ATCC PCS-201-012), dimethylmethylene blue (Sigma-Aldrich), alcian blue (Tyr Scientific LLC, 66075), paraformaldehyde (Sigma-Aldrich), FITC Conjugation Kit (abcam), Alexa Fluor™ 555 goat-anti-rabbit (invitrogen), Alexa Fluor™ 546 goat anti-mouse IgG1 (y1) (invitrogen), Alexa Fluor™ 488 phalloidin (A12379, invitrogen), NuclearBlue (Cell Signal) PrestoBlue™ Cell Viability Reagent (A13262, invitrogen), Fluorescein isothiocyanate-dextrane (FD40S-100 MG, invitrogen), Pecicillin Streptomycin (gibco), 0.25% Trypsin-EDTA (1×) (gibco), sodium hyaluronate (gibco), LIVE/DEAD™ Cell Imaging kit (R37601, invitrogen), Dulbecco's Modification of Eagle's Medium (DMEM) (Corning. 10-013-CV, Lot 21621002), 35 mm glass bottom dishes (MatTek). MatTek dishes with silica glass wells were cleaned in preparation for surface functionalization. The dishes underwent a DI H₂O was with 10 minutes of sonication. A IM HCl treatment followed, and the dishes were again sonicated for 10 minutes before two rinses with DI H₂O. Separate pure 2-propanol and ethanol treatments with 10 minute sonication periods followed. After sonication with these treatments, the solvents were aspirated, and the dishes were allowed to dry before two washes with DI H₂O. The dishes were sonicated for 10 minutes with DI H₂O, and the DI H₂O was aspirated from the dishes.

MatTek dishes with silica glass wells were cleaned in preparation for surface functionalization. The dishes underwent a DI H₂O was with 10 minutes of sonication. A IM HCl treatment followed, and the dishes were again sonicated for 10 minutes before two rinses with DI H₂O. Separate pure 2-propanol and ethanol treatments with 10 minute sonication periods followed. After sonication with these treatments, the solvents were aspirated, and the dishes were allowed to dry before two washes with DI H₂O. The dishes were sonicated for 10 minutes with DI H₂O, and the DI H₂O was aspirated from the dishes.

Surface functionalization was done with 2% APTMS and 1 mM HABP. 2% APTMS (in 2-propanol) was treated on the surface for 6 hours in a wet chamber at room temperature. Following the end of the culture period, three washes with DI H₂O were used to remove the residual APTMS. A 1 mM HABP in DI H₂O solution was added to the surface and left overnight at 4° C. in the wet chamber. After the peptide treatment is completed, the surfaces were rinsed 5 times with PBS for 5 minutes each. These functionalized surfaces were used for HA binding verification and cell seeding experiments. Two other experimental groups were established: one with only the APTMS treatment, and one with no surface treatment (Table 2).

TABLE 2
Experimental groups and their surface treatments

| | | |
|---|---|---|
| Untreated | APTMS | HABP |
| Untreated | Surface treated with 2% | Surface treated with |
| Surface | APTMS | GAHWQFNALTVR (1 mM) (SEQ |
| | | ID NO: 1) |

Human primary dermal fibroblasts (HDFs) were cultured in a culture media consisting of DMEM, 10% fetal bovine serum (FBS), and 1% penicillin-streptomycin (10,000 IU/mL). The cells were rinsed with warm 1×PBS to remove any traces of FBS from the surface, and a 0.25% trypsin/EDTA treatment was used to detach the cells from the culture flask at 37° C. for 5 minutes. 4 mL of fresh media was added to the cells to inhibit the trypsin, and the suspended cells were withdrawn from the flask, counted using a hemocytometer, and added to a 15 ml centrifuge tube. The cells were centrifuged for 5 minutes at 1500 rpms, and the media was withdrawn and replaced with a calculated amount of fresh media in which the cells were resuspended. The cells were added to the functionalized MatTek surfaces, and after 6 hours, 2 mL of media containing 100 μg/mL ascorbic acid was added. The cells were cultured for 48-96 hours before further experimentation was conducted.

### Immunostaining for Type I Collagen and α-SMA

Immunostaining was done using α-SMA (D4K9N, Cell Signal) and Type I collagen primary antibodies. The cells were first rinsed with warm PBS (37° C.) for 5 minutes to remove media residue from the surface. The cytosol was removed from the cells using ice cold cytoskeletal stabilization buffer (50 mM Sodium chloride, 0.5% Triton X-100, 10 mM PIPES, 2.5 mM magnesium chloride, 1 mM EGTA, 0.3M sucrose, protease inhibitors, phosphatase inhibitors) for 1 minute followed immediately by fixation with 3.7% paraformaldehyde for 15 minutes. PBS was used to rinse 3 times for 15 minutes before the cultures were treated with permeabilization buffer (PB) (1×PBS, 2% bovine serum albumin, 0.1% Triton X-100) for 45 minutes. Diluted primary antibody in PB was added to the cells and cultured overnight at 4° C. and then washed again with the same series of PBS washes. After this point, all further staining was conducted in the dark to preserve fluorescence. Fluorescently conjugated Secondary antibody in PB (Alexa Fluor 555 (1:200)) was then introduced to the cells and cultured for 45 minutes followed by Alexa Fluor 488 Phalloidin (lot number, company catalog number, ratio) dye in PB for 30 minutes and Nuclear Blue in PB (lot, company, catalog, 1:1000) for 5 minutes with PBS washes between each stain. The cells were then imaged using fluorescent microscopy.

### Results and Discussion

### Cytocompatibility of HABP Functionalized Surfaces

PrestoBlue is a cytocompatibility assay reagent that uses resazurin as an active ingredient. When resazurin enters live cells and is exposed to NADPH, it is reduced to resorufin, which then fluoresces red. With higher amounts of metabolic activity, more fluorescence is expressed. The fluorescence can be read with a plate reader and produce quantifiable cell viability data.

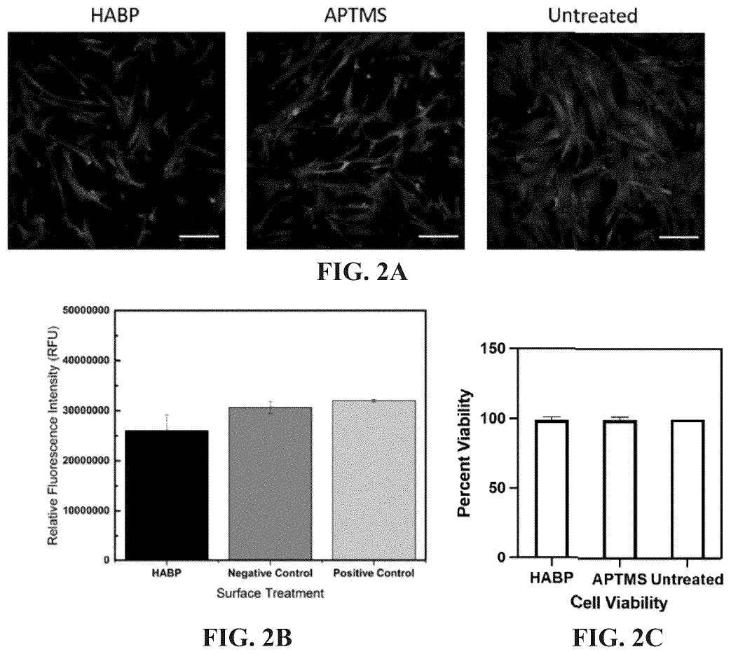

The scrambled HABP sequence was used to compare to the normal HABP surface functionality. The results of the assay determined that the solubilized peptide had no significant effect on the cell viability. While the viability did decrease in peptide treated experimental groups, especially at higher peptide concentrations, the overall fibroblast viability was still high enough compared to the controls (FIG. 2).

Live/Dead fluorescent staining was done to determine the viability of fibroblasts cultured on the functionalized surfaces. While HABP had a slightly lower viability than the APTMS treated and untreated surfaces, the fibroblasts still had a viability of 98.9%. As seen in FIG. 2A, cells were highly viable in all experimental groups. Additionally Presto Blue experiment was performed to assess metabolic activity of fibroblasts on peptide treated surfaces FIG. 2B There was no significant difference between percent viability as depicted in FIG. 2C. These results indicate that HABP is cytocompatible.

### Cell Morphology on HABP Functionalized Surfaces

APTMS functionalized and untreated experimental groups were established. The APTMS functionalized surfaces were treated with 2% APTMS only and the other experimental group did not undergo any treatment.

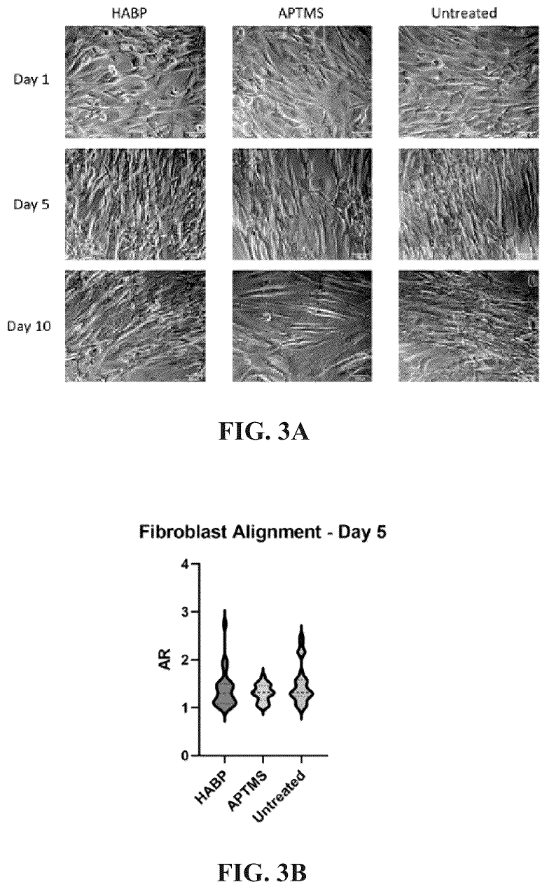

Cells seeded on the APTMS treated surfaces presented with a slightly stressed morphology, but overall had no significant differences in morphology from cells on Untreated and HABP surfaces (FIG. 3). The distribution of fibroblast alignment was found to be similar between all three treatment groups. Untreated and HABP functionalized surfaces did have some fibroblasts with higher levels of alignment, but the majority of fibroblast cells in those groups maintained a similar distribution of alignment to the APTMS cultures.

In low cell seeding densities, the fibroblasts died, while in higher seeding densities, they grew in sparser colonies than the other experimental groups but did not completely die off. The poor performance of the fibroblast cells in the APTMS functionalized group was attributed to the APTMS on the surface and the high concentration of amine groups available for binding creating a negative environment for cell growth and development. Increasing the number of PBS washes following the peptide treatment in dish preparation and conditioning the dish with culture media before cell seeding improved cell growth in the APTMS treated group in further cultures. In the untreated cultures, cells grew normally and had high surface coverage.

Culture time was optimized as well. In early cultures cells became contractile and began to lift off the dish by day 6 or 7. This led to decellularization being required earlier than desired. Previous literature indicated that a culture time of 5-9 days was ideal for FDM deposition. With the selected seeding density of 50,000 cells/154 mm2, a culture time of 11 days was initially found to be ideal for matrix deposition. After surface cleaning was introduced to the protocol, it was noted that by day 10, the cells had become contractile, therefore pulling on the deposited matrix. Following the decellularization procedure, the FDMs lifted off with the cell debris, leaving behind minimal matrix. This difference in cell behavior could be attributed to the reduction in surface topography due to the elimination of the impurities. After evaluating the cell alignment and area data, it was proposed that 11 days in culture was too long, and a shorter culture time may yield better results.

### Fibroblast Activity on HABP Functionalized Surfaces

Fibroblast cells are responsible for secreting and organizing ECM components in the body, and upregulation of fibroblast activity has been linked to fibrosis through excessive ECM deposition. Among the ECM components secreted by fibroblasts is HA. In order to study the ECM components deposited by fibroblasts, a method of producing three-dimensional (3D) fibroblast derived matrices (FDMs) was developed. In culture, fibroblasts are cultured at a confluent state for several days. At the end of this time period, the cells are exposed to a decellularization agent that leaves behind the deposited matrix. This protocol was used to study the effect of HABP functionalized surfaces on ECM deposition by fibroblast cells.

Initial cell culture experiments for FDM deposition focused on optimization of cell seeding density for best matrix deposition. Cell seeding densities of 20,000, 40,000, 50,000, and 60,000 cells/154 mm² were evaluated. Cell alignment and cell coverage area were evaluated at this time to determine if and how they were affected by the functionalized surfaces. The lower seeding densities did not deposit high levels of matrix, and while 60,000 cells/154 mm² deposited more matrix, using a seeding density of 50,000 cells/154 mm² allowed for a longer time in culture and adequate matrix deposition and was therefore the selected seeding density for further culture.

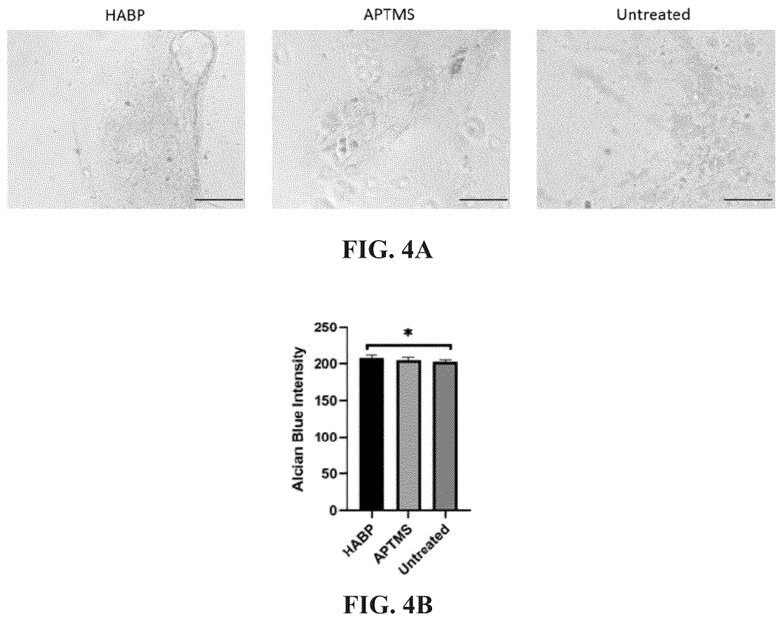

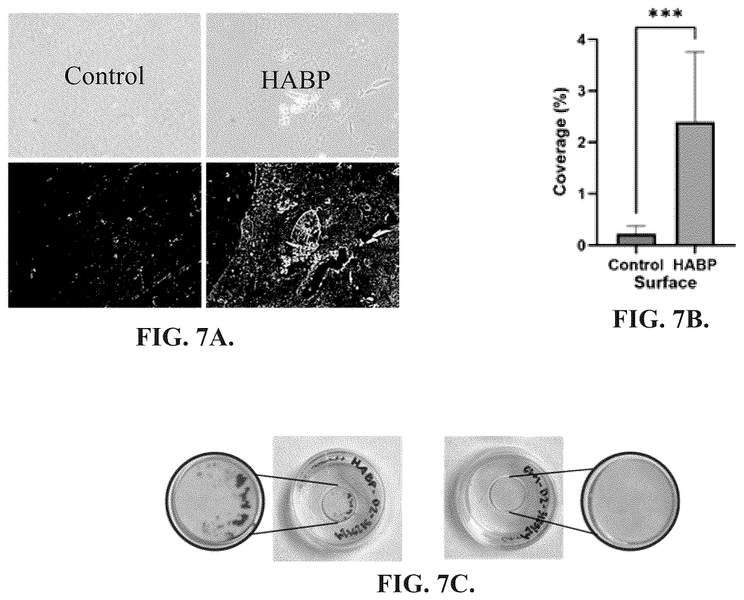

Alcian Blue (AB) staining was used to evaluate whether the HABP surface functionalization influenced the amount of HA deposited within the FDMs compared to Scrambled HABP and the two controls. Darker, more specific staining was noted on the peptide treated surfaces compared to the controls. HABP treated surfaces had an intensity 1.55% higher than the APTMS treated surfaces and 2.43% than the untreated surfaces. The HABP treated surfaces were found to have a higher AB intensity. As can be seen in FIG. 4A, blue staining can be noted on matrices in the peptide group, while minimal specific staining is seen in the controls. When the data was quantified using the mean intensity function of imageJ, it was noted that results largely matched qualitative observation; as can be seen in FIG. 4B the intensity values of the peptide cultures were higher than that of the other surfaces. These results indicate that HABP was successful at inducing additional HA deposition from the fibroblast cells. Utilizing the same method and materials. Alcian Blue (AB) staining was used to evaluate the HA binding ability of HABP. As can be seen in FIG. 7A, blue staining can be noted on the HABP treated surfaces. The HABP treated surface was found to have a higher Coverage (%) than the Control surface as can be seen in FIG. 7B and FIG. 7C. These results indicate that HABP treated surfaces attract more stain after being treated with HA. HA confirming HABP to attract HA.

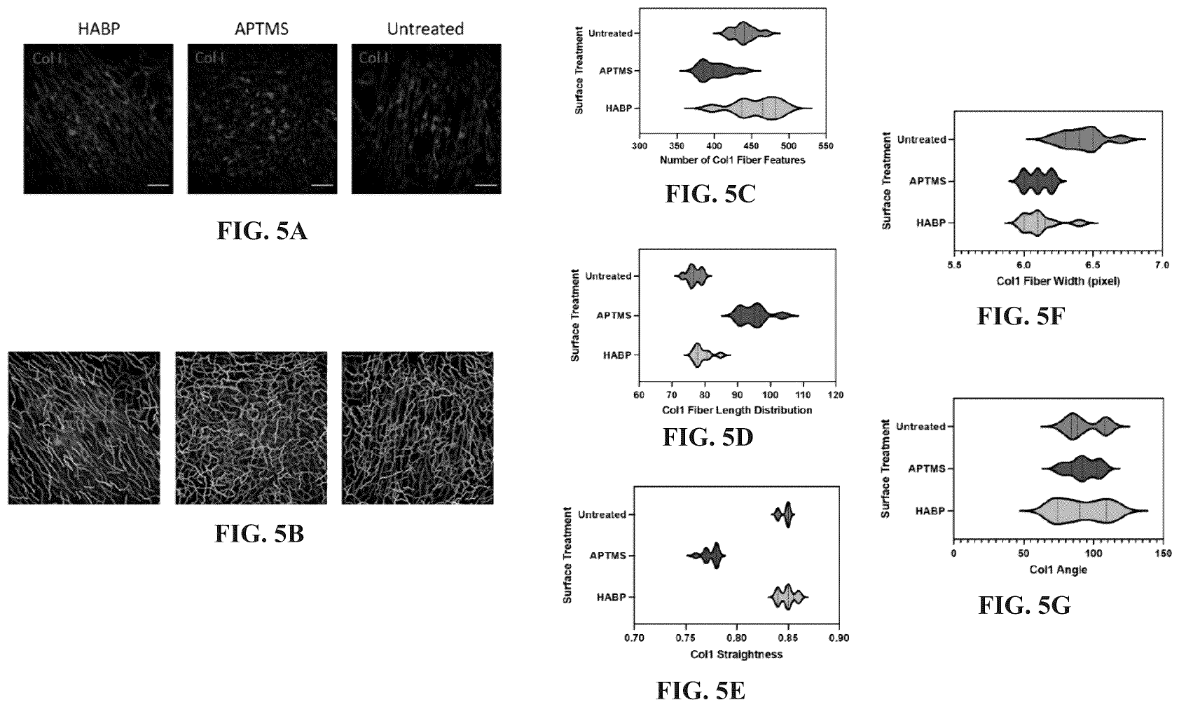

Immunostaining for Type I Collagen (Col1) was done to determine the impact of the HABP surface treatment on ECM formation. As can be seen in FIG. 5A and FIG. 5B there was a clear difference between the experimental groups, with collagen fibers in the APTMS experimental group appearing more immature, as well as the collagen being concentrated within the cells in the APTMS and Untreated cultures, while the fibers were outside of the cell in the HABP cultures. The HABP surface functionalization had an impact on ECM deposition that included the rate at which the Col1 was deposited. Col1 fibers were analyzed and the analysis found significant differences in fiber angle, straightness, and width. The collagen fibers in the HABP and APTMS cultures had a width 5% narrower than those of the untreated cultures with a significance of p-value<0.01.

This result indicated that the APTMS functionalization of the surface played some role in the reduction of collagen width because the same results were not seen in the untreated surfaces. In FIGS. 5C, D, E, F, the violin plot distribution is also clearly different when comparing the Untreated cultures to the fibers in the APTMS and HABP cultures. Regarding length, the fibers in the HABP cultures were shorter than the APTMS surfaces, but not to a significant degree. The fibers were just 5% shorter on the HABP treated surfaces compared to the APTMS cultures. The Col1 fibers in the untreated cultures were also shorter than those in the APTMS surfaces, indicating that the APTMS treatment alone may have some role in affecting Col1 fiber length.

FIG. 5D provides a clear picture of the differences in length between the treatment groups. APTMS has both a wider distribution of fiber lengths and greater length than the other two groups of cultures. The straightness of the fibers was significantly higher (p-value<0.001) in the HABP indicating that the HABP surface functionalization had some impact on the Col1 fiber development. As can be seen in FIG. 5A, the Col1 fibers in the APTMS experimental group appear more immature than those in the other two cultures. While there were determined to be a higher number of Col1 fibers in the HABP cultures compared to the two other experimental groups, the fibers were narrower and shorter with respect to the controls specified above. This indicates that the HABP treatment has a significant effect on Col1 deposition by fibroblast cells. The angle of the fibers was higher in the Untreated cultures, indicating that these fibers were more aligned than in HABP and Untreated cultures.

Overall, these results indicate that the Untreated cultures were more aligned and thicker than the HABP cultures, meaning that the fibers in these cultures were more well-developed than the treated cultures, indicating that the treated cultures have reduced collagen fiber formation.

### Actin Activity on HABP Functionalized Surfaces

Actin is a cytoskeletal protein responsible for cell movement and maintaining cell structure. Immunostaining was used to visualize actin filaments in living cells. The actin and their adhesions were evaluated to determine if there was a discernible difference in actin behavior between HABP functionalized surfaces and the controls. ImageJ was used to quantify the fluorescent intensity and coverage area of actin filaments.

The same experimental groups were stained for actin again along with staining with DAPI for the nucleus of the fibroblast cells so that a representation of the number of cells could be analyzed in line with the actin filament behavior. In this case, actin had a higher amount of culture coverage in the Untreated experimental groups, but there was no statistical significance in the area covered in any experimental group. The untreated surfaces also had a higher fluorescent intensity than HABP and the APTMS treatment group, but again there was no statistical significance between the three experimental groups.

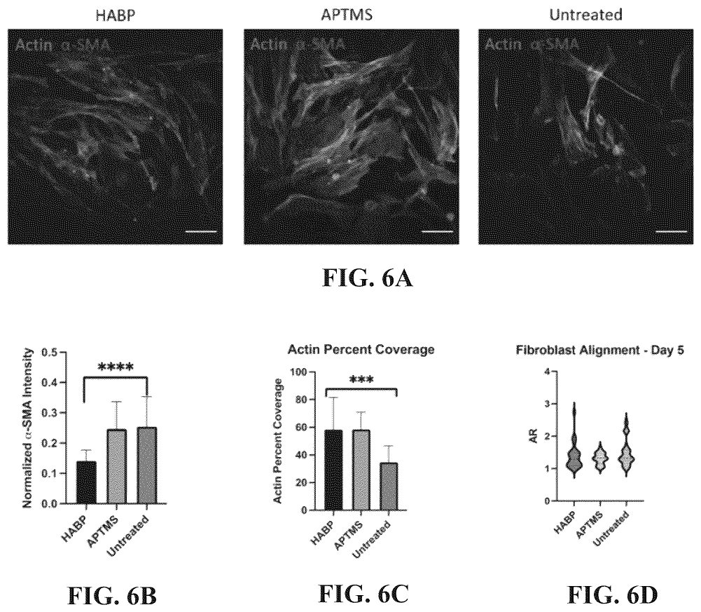

These studies have shown that at 48 hours of cell culture, there are no significant morphological differences observed impacted by surface functionalization. Further immunostaining for α-SMA (Cell Signal, Lot 3, D4KN9) and F-actin (phalloidin) in fibroblast cells after 48 hours in culture revealed that cell adhesion was lower in untreated surfaces compared to the treated surfaces of the HABP and APTMS. Actin plays a role in cell structure and motility, and myofibroblasts are characterized by higher amounts of actin stress fibers. Actin with more clearly defined stress fibers was noted in the controls compared to the HABP cultures, leading to the conclusion that myofibroblast differentiation and contraction were reduced on HABP-treated surfaces. Significantly less actin was noted in the untreated cultures, indicating lower cell adhesion in those cultures, which was determined by a lower cell number (indicated with Nuclear Blue staining) and less actin surface coverage. α-SMA is an actin protein that has been linked to myofibroblast formation and associated with the progression of fibrosis. α-SMA in fibroblast cultures is also an indication of contractile cells. Nearly 45% less α-SMA signal was detected in the HABP cultures compared to the controls, indicating that the surface functionalization with HABP had a protective effect on fibroblast differentiation into myofibroblasts, as well as the contractile phenotype of the cells. As seen in FIG. 6, little α-SMA signal was detected in the HABP cultures, while it was present in both controls, demonstrating the effect of HABP. Normalized intensity measurements of the α-SMA to the total F-actin signal indicate that the signal in the HABP cultures was significantly lower than that of the controls with a p-value of 0.00038.

### CONCLUSIONS

In the evaluation of Alcian Blue and immunostaining studies, it was found that HABP functionalized surfaces decreased α-SMA presence in fibroblast cell cultures, increased HA deposition from fibroblast cells, and altered collagen fiber geometry and deposition. The experimental data gave a clear visible indication that Col1 fibers produced by fibroblast cells cultured on HABP functionalized surfaces were more mature than APTMS and Untreated cultures. HA plays a critical role in regenerative wound healing with its ability to influence ECM deposition.

From this study, it can be concluded that HABP does contribute towards increased ECM regulation. Col1 fibers reacted differently from controls in cultures with HABP functionalized surfaces, producing narrower and shorter fibers compared to the controls, which indicates that additional HA being attracted to the surface was regulating the deposition of the Col1 fibers of the ECM. Our results indicated treatment of surfaces with HABP increased HA deposition and diminished α-SMA expression of primary human dermal fibroblasts. Furthermore, the Col1 fibrils deposited on HABP-treated surfaces exhibited increased organization and decreased fibril lengths indicating further investigation to be done to understand regulatory mechanisms fibroblast deposited HA play on collagen assembly on HABP-treated surfaces.

The disclosure being thus described, it will be obvious that the same may be varied in many ways. Such variations are not to be regarded as a departure from the spirit and scope of the disclosures and all such modifications are intended to be included within the scope of the following claims. The above specification provides a description of the manufacture and use of the disclosed treated articles and related methods. Since many embodiments can be made without departing from the spirit and scope of the disclosure, the invention resides in the claims.

What is claimed is:

1. A method of decreasing inflammation and/or promoting wound healing in a patient, comprising:
providing to the patient a biomaterial comprising a hyaluronic acid binding peptide (HABP), wherein the HABP increases hyaluronic acid (HA) deposition in an area surrounding the biomaterial.

2. The method of claim 1, wherein the HABP is bound to a surface functionalizing agent.

3. The method of claim 2, wherein the surface functionalizing agent is an aminoorganosilane, a maleimide, a thiol-maleimide, a glutaraldehyde, a dopamine, a hydroxypyrrolidine, a diisothiocyanate, a catechol, a polyester, and/or an aldehyde.

4. The method of claim 2, wherein the surface functionalizing agent is an aminoorganosilane.

5. The method of claim 4, wherein the aminoorganosilane is 3-aminopropyltrimethoxysilane (APTMS).

6. The method of claim 1, wherein the biomaterial is a liquid, gel, hydrogel, wound dressing, wound mesh, surgical mesh, nanofiber, and/or suture.

7. The method of claim 1, wherein the HABP comprises the amino acid sequence of any one of SEQ ID NOs: 1, 2, 3, 4, or 5.

8. The method of claim 1, wherein the HABP comprises the amino acid sequence of SEQ ID NO: 1.

9. The method of claim 1, wherein said providing step comprises injecting the biomaterial into the skin or a tissue of the patient.

10. The method of claim 1, wherein said providing step comprises topical application of the biomaterial.

11. The method of claim 2, wherein the biomaterial is an implant or medical device.

12. The method of claim 11, wherein at least one surface of the implant or medical device is coated with the surface functionalizing agent bound to the HABP.

13. The method of claim 1, wherein the biomaterial improves and/or reduces a foreign body reaction.

14. The method of claim 13, wherein said foreign body reaction comprises fibrotic capsule formation.

15. The method of claim 1, wherein fibroblast cells in an area surrounding the biomaterial exhibit increased collagen fiber organization, decreased collagen fiber length, and/or reduced collagen fiber formation.

16. An article comprising:
a coating of a surface functionalizing agent on a surface of the article, wherein a hyaluronic acid binding peptide (HABP) is bound to the surface functionalizing agent;
wherein the surface functionalizing agent is selected from the group consisting of an aminoorganosilane, a maleimide, a thiol-maleimide, a glutaraldehyde, a dopamine, a hydroxypyrrolidine, a diisothiocyanate, a catechol, a polyester, and an aldehyde.

17. The article of claim 16, wherein the surface functionalizing agent is an aminoorganosilane.

18. The article of claim 16, wherein said article is an implant or medical device.

19. The article of claim 16, wherein the HABP comprises the amino acid sequence of any one of SEQ ID NOs: 1, 2, 3, 4, or 5.

20. A method of improving and/or reducing foreign body reaction to an article, comprising:
coating at least one surface of the article with a surface functionalizing agent, wherein a hyaluronic acid binding peptide (HABP) is bound to the surface functionalizing agent to form a treated article; and
providing the treated article to a patient, wherein said providing comprises at least partially implanting the treated article within the patient.
